(19) United States
(12) Patent Application Publication
(10) Pub. No.: US 2026/0284117 A1
(43) Pub. Date: Sep. 24, 2026

(54) METHODS FOR TREATING POLYQ DISEASE AND NOVEL ADIPOSE TISSUE DERIVED MESENCHYMAL STEM CELLS

(71) Applicant: STEMINENT BIOTHERAPEUTICS INC., Taipei (TW)

(72) Inventors: Wei-Chi LO, Taipei (TW); Wei-Kee ONG, Taipei (TW); Hsiu-Yu LAI, Taipei (TW)

(21) Appl. No.: 19/484,405

(22) PCT Filed: May 15, 2024
(86) PCT No.: PCT/CN2024/093397

§ 371 Date: Nov. 14, 2025

(30) Foreign Application Priority Data
May 15, 2023 (AU) 2023901470

(51) Int. Cl. A61K 35/28; A61K 38/18; A61P 25/28

(52) U.S. Cl. CPC A61K 35/28; A61K 38/18; A61P 25/28

(57) ABSTRACT

Provided is the use of adipose tissue derived stem cells that express Activin A or the combination of Activin A and modified adipose tissue derived stem cells, for treating polyQ diseases, such as spinocerebellar ataxia 3 (SCA 3), by reducing a misfolded and aggregated polyQ protein, such as ataxin-3 protein. Also provided is a composition for reducing misfolded and aggregated polyQ proteins, wherein the composition comprises a population of modified adipose tissue derived stem cells and Activin A.

### CROSS-REFERENCES TO RELATED APPLICATION

This application claims the benefit of priority from Australian Provisional Application No. 2023901470, filed 15 May 2023, the entire content of which is incorporated herein by reference.

### BACKGROUND

There are nine inherited neurodegenerative disorders, collectively known as polyglutamine (polyQ disease), including Huntington's disease (HD), dentatorubral-pallidoluysian. atrophy (DRPLA), spinal bulbar muscular atrophy (SBMA), and spinocerebellar ataxias (SCA) 1, 2, 3, 6, 7 and 17. The polyQ diseases are caused by a polyglutamine (polyQ) expansion in their respective disease proteins and share a common pathological feature: the nuclear accumulation of polyQ disease protein and the formation of inclusions.

The most common pathogenic cascade of the polyQ disease is the misfolding and aggregation of the expanded polyQ proteins (T. Takeuchi et al. “Disease protein misfolding and aggregations a therapeutic target for polyQ diseases” Brain Science 2017:128, M. Arrasate et al. “Protein aggregates in Huntington's disease” Exp Neuro 20-12; 1-11, A. Klement et al. “Ataxin-1 Nuclear Localization and Aggregation: Role in Polyglutamine-Induced Disease in SCA1 Transgenic Mice” Cell 1998:41, M Mark et al “Spinocerebellar Ataxia Type 6 Protein Aggregates Cause Deficits in Motor Learning and Cerebellar Plasticity” The Journal of Neuroscience, 2015:8882-8895). To date, there is no definitive treatment to cure or slow the progression of polyQ diseases.

There is a need for reducing mutant polyQ proteins and an effective treatment for polyQ diseases. The present invention addresses this need and other needs.

### BRIEF DESCRIPTION OF THE INVENTION

In one embodiment, the present invention provides methods for treating polyQ diseases, comprising the step of administering a therapeutic effective amount of (a) Activin A; and (b) a population of modified adipose tissue derived stem cells (modified ADMSCs) with phenotypic characteristic of CD273, CD46, CD55, CXCR4, CD90, CD105 and CD73 but without expressing CD45, CD34, CD11b, CD19 and HLA-DR), and maintain osteogenic, chondrogenic and adipogenic differentiation capacities, to a subject in need thereof.

The present invention also provides methods for treating polyQ disease, comprising the step of administering a therapeutic effective amount of the Activin A-expressing ADMSCs described herein to a subject in need thereof, said Activin A-expressing ADMSCs have the phenotypic characteristic of CD273, CD46, CD55, CXCR4, CD90, CD105 and CD73 (but without expressing CD45, CD34, CD11b, CD19 and HLA-DR).

Also provided are methods for reducing a mutant polyQ protein (for example, ATXN-3) in a subject, comprising the step of administering a therapeutic effective amount of (a) Activin A; and (b) modified ADMSCs described herein, to a subject in need thereof.

Also provided are methods for reducing a mutant polyQ protein (for example, ATXN-3) in a subject with polyQ disease, comprising the step of administering a therapeutic effective amount of Activin A-expressing ADMSCs described herein, to a subject in need thereof.

Some embodiments provide an isolated population of modified ADMSCs that express Activin A (Activin A-expressing ADMSCs), said Activin A expressing-ADMSCs express adipose tissue derived stem cell markers, comprising CD90, CD105. and CD73 but not CD45, CD34, CD11b, CD19 and HLA-DR, maintain osteogenic, chondrogenic and adipogenic differentiation capacities.

Other embodiments provide pharmaceutical compositions comprising at least one Activin A ADMSCs described herein and a pharmaceutically acceptable carrier or excipient.

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification, any or all drawings and each claim.

The invention will become more apparent when read with the accompanying figures and detailed description which follow.

### BRIEF DESCRIPTION OF THE DRAWINGS

Illustrative embodiments of the present invention are described in detail below with reference to the following Figures:

FIG. 1 is a bar graph illustrating Activin A reduces mutant ataxin-3 level in the SH-SY5Y ATXN3/Q75 cells in a dose dependent manner.

FIG. 2A and FIG. 2B show the synergistic effect of the combinations of various doses of modified ADMSC and various doses of Activin A in reducing mutant ataxin-3 (The CI value is less than 1 indicates synergy).

FIG. 3 is a line graph showing that conditioned medium from Activin A expressing ADMSCs reduces mutant ataxin-3 expression in a dose dependent manner, compared to that of plasmid control group.

FIG. 4A to FIG. 4C are bar graphs showing the effect of different concentrations of Activin A, on the proliferation of modified ADMSCs seeded at different densities.

FIG. 5A and FIG. 5B are flow cytology images of the native ADMSCs isolated from adipose tissue.

### DETAILED DESCRIPTION OF THE INVENTION

As used herein, the articles “a” and “an” refer to one or more than one (i.e., at least one) of the grammatical object of the article. By way of example, “amodified ADMSC” means one modified ADMSC or more than one modified ADMSCs.

A “therapeutic effective amount,” as used herein, refers to a dose of the modified ADMSCs or the pharmaceutical composition disclosed herein that is sufficient to result in reduction of mutant polyQ protein (for example, ATXN-3) or improvement in one or more symptoms and signs of a polyQ disease, which include, but are not limited to, ataxia, involuntary movements, cognitive impairment, which are detectable, either clinically or radiologically through various imaging means.

The term “treating,” “treated,” or “treatment” as used herein refers to palliative uses or results, and/or slowing or inhibiting the advancement of a polyQ disease, the formation of mutant polyQ protein, neuronal intracellular inclusions.

The term “subject” can refer to a vertebrate having or suspected of having a polyQ disease or to a vertebrate deemed to be in need of treatment for a polyQ disease. Subjects include warm-blooded animals, such as mammals, a primate, and, more preferably, a human. Non-human primates are subjects as well. The term subject includes domesticated animals, such as cats, dogs, etc., livestock (for example, cattle, horses, pigs, sheep, goats, etc.) and laboratory animals (for example, mouse, rabbit, rat, gerbil, guinea pig, etc.). Thus, veterinary uses and medical formulations are contemplated herein.

The expression level or surface density of a surface marker, such as CD273, on the surface of the modified ADMSCs are “+” as long as the expression of the surface marker is detectable by any conventional means. In one exemplary embodiment, “+” means that the cellular surface marker is detectably present in fluorescence activated cell sorting/flow cytometry or magnetic beads over an isotype control; or is detectable above background in quantitative or semi-quantitative RT-PCR.

The expression level or surface density of a surface marker, such as CD45, is “−” as long as the expression of the surface marker is not detectable by any conventional means. In one exemplary embodiment, “−” means that the cellular surface marker is not detectably present in fluorescence activated cell sorting/flow cytometry or magnetic beads over an isotype control; or is not detectable above background in quantitative or semi-quantitative RT-PCR.

As used herein, “substantially free” means less than 2%, 1% or 0.5%.

All numbers herein may be understood as modified by “about.” As used herein, the term “about” is meant to encompass variations of +10%.

The term “adipose tissue derived mesenchymal stem cell” and “adipose tissue derived stem cell” are used interchangeably. The abbreviation “ADMSC” applies to both “adipose tissue derived mesenchymal stem cell” and “adipose tissue derived stem cell”.

### Modified ADMSCs

Modified ADMSCs of the present invention are derived from native mesenchymal stem cells isolated from adipose tissue. The native mesenchymal stem cells isolated from adipose tissue (native ADMSCs) are characterized as CD105⁺, CD73⁺, CD90⁺, CD45⁻, CD34⁻, CD11b⁻, CD19⁻, HLA-DR⁻, CD273⁻ and maintain osteogenic, chondrogenic and adipogenic differentiation capacities. In one embodiment, the native ADMSCs are CD46⁻, CD55⁻, CXCR4⁻.

A population of the modified ADMSCs described herein is considered a purified population of modified ADMSCs if less than 2% of the ADMSCs express CD45, CD34, CD11b, CD19 and HLA-DR.

Example 1 of the present application illustrates one embodiment of the culture method to generate the modified ADMSCs of the present invention, wherein the native ADMSCs were cultured in a MSC culture medium for about 12 hours to about 7 days, wherein the MSC culture medium comprises alpha-MEM, UltraGRO and Heparin. In another embodiment, the MSC culture medium comprises epidermal growth factor (EGF), fibroblast growth factor 2 (FGF-2), and insulin-transferrin-selenium (ITS) supplement.

In one embodiment, the modified ADMSC is characterized by the expression of CD273, CD46, CD55, CXCR4, which are not present in naturally occurring or native ADMSCs. The phenotypic characteristic of the modified ADMSCs is CD273⁺, CD46⁺, CD55⁺, CXCR4⁺, CD105⁺, CD73⁺, CD90⁺, but not CD45⁻, CD34⁻, CD11b⁻, CD19⁻, HLA-DR. The modified ADMSCs maintain osteogenic, chondrogenic and adipogenic differentiation capacities.

### Activin A Expressing ADMSCs

In one embodiment, one or more Activin A plasmids are inserted into the modified ADMSCs described herein. The modified ADMSCs can express Activin A and are known as Activin A expressing ADMSCs.

The modified ADMSCs and Activin A expressing ADMSCs of the present invention are non-naturally occurring, as native ADMSCs do not express CD273.

The modified ADMSCs can be from a single individual, i.e., autologous, or pooled from multiple individuals (non-autologous allogeneic).

### Pharmaceutical Composition

In one embodiment, a pharmaceutical composition comprising at least one modified ADMSC described herein, activin A and a pharmaceutically acceptable vehicle, carrier or excipient is provided.

Advantageously, this combination has synergistic effects on the reduction of mutant polyQ protein (for example, ATXN-3) or improvement in one or more symptoms and signs of a polyQ disease.

Suitable vehicles are, for example, water, saline, dextrose, glycerol, ethanol, Dimethyl sulfoxide (DMSO), trehalose, and combinations thereof. Suitable excipients are, for example, wetting agent, emulsifying agents or pH buffering agents. Pharmaceutically acceptable carriers can contain a physiologically acceptable compound that acts to, e.g., stabilize, or increase or decrease the absorption or clearance rates of the pharmaceutical compositions of the invention. Physiologically acceptable compounds can include, e.g., carbohydrates, such as glucose, sucrose, or dextrans, antioxidants, such as ascorbic acid or glutathione, chelating agents, low molecular weight proteins, detergents, liposomal carriers, or other stabilizers and/or buffers. Other physiologically acceptable compounds include wetting agents, emulsifying agents, dispersing agents or preservatives. The excipients may be nonionic surfactants, polyvinylpyrollidone, human serum albumin, aluminum hydroxide, agents with anesthetic action, and various unmodified and derivatized cyclodextrins. In one embodiment, the nonionic surfactants may include Polysorbate 20, Polysorbate 40, Polysorbate 60, and Polysorbate 80. The polyvinylpyrollidone may preferably be Plasdone C15, a pharmaceutical grade of polyvinylpyrollidone. The pharmaceutical composition comprising such excipient or carrier are formulated by well-known conventional methods.

In an exemplary embodiment, the pharmaceutical composition is substantially free of bone marrow-derived mesenchymal stromal cells.

The invention also provides methods of treating a polyQ disease, by administering to a subject in need thereof a therapeutic effective amount of the pharmaceutical composition described herein or Activin A expressing ADMSC described herein. The polyQ disease includes Huntington's disease (HD), dentatorubral-pallidoluysian atrophy (DRPLA), spinal bulbar muscular atrophy (SBMA), and six spinocerebellar ataxias (SCA1, SCA2, SCA3, SCA6, SCA7, and SCA17).

In some embodiment, methods for reducing the mutant polyQ protein (for example, ATXN-3) are provided, by administering to a subject in need thereof a therapeutic effective amount of the pharmaceutical composition described herein or Activin A expressing ADMSC described herein. In one embodiment, the polyQ protein is ATXN-3 for SCA3.

In a further embodiment of the present invention, the use of the modified ADMSCs and Activin A combination, or the Activin A expressing ADMSC, as described herein, for the manufacture of a medicament for the treatment of a polyQ disease or reducing the disease polyQ protein is provided.

Routes of administration of the present pharmaceutical compositions, of the modified ADMSCs and Activin A combination and of Activin A expressing ADMSCs include, but are not limited to, intravenous, intramuscular, subcutaneous, oral, topical, subcutaneous, intradermal, transdermal, subdermal, parenteral, rectal, spinal, intrathecal, intraventricular, intracranial or inter-vertebrate administration. In one embodiment, the present pharmaceutical compositions, the modified ADMSCs and Activin A combination or Activin A expressing ADMSCs are administered by intravenous injection or infusion.

The modified ADMSCs of the present invention can be administered simultaneously, before or after Activin A for treating polyQ disease.

The Activin A expressing ADMSCs or the pharmaceutical compositions described herein can be administered in a single dose treatment or in multiple-dose treatments on a schedule and over a time period appropriate to the age, weight and condition of the subject, the particular composition used, and the route of administration, whether the Activin A expressing ADMSCs or the present pharmaceutical composition is used for prophylactic or curative purposes, etc. In some embodiments, the Activin A expressing ADMSCs or the present pharmaceutical composition is administered once a year, once every 6 months, once every 4 months, once every 3 months, once per month, twice per month, three times per month, once every other week (qow), once per week (qw), twice per week (biw), three times per week (tiw), four times per week, five times per week, six times per week, every other day (qod), daily (qd), twice a day (qid), or three times a day (tid).

The data obtained from the cell culture assays and animal studies can be used in formulating a range of dosages for use in humans. In one embodiment, the dosage lies within a range of circulating concentrations that include the ED50 with little or no toxicity. The dosage can vary within this range depending upon the dosage form employed and the route of administration utilized. In another embodiment, the therapeutically effective dose can be estimated initially from cell culture assays. A dose can be formulated in animal models to achieve a circulating plasma concentration range that includes the IC50 (i.e., the concentration which achieves a half-maximal inhibition of symptoms) as determined in cell culture. Sonderstrup, Springer, Sem. Immunopathol. 25:35-45, 2003. Nikula et al., Inhal. Toxicol. 4 (12): 123-53, 2000.

The pharmaceutical composition is formulated to contain a therapeutic effective amount of the modified ADMSCs described herein and Activin A, or Activin A expressing ADMSCs, wherein the amount depends on the subject to be treated and the condition to be treated. The specific dose level for any particular subject depends upon a variety of factors including the activity of the specifically modified monocytes or modified ADMSCs, the age, body weight, general health, sex, diet, time of administration, route of administration, and rate of excretion, drug combination and the severity of the particular disease undergoing therapy. An exemplary, non-limiting range for a therapeutically or prophylactically effective amount of the modified ADMSCs/Activin A Expressing ADMSCs of the present invention is at least about 1×10⁴ cells per dose to about 1×10¹⁰ per dose. Other dosages are also possible, including, but not limited to, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸ or 1×10⁹, 2×10⁹, 3×10⁹, 4×10⁹, 5×10⁹, 6×10⁹, 7×10°, 8×10⁹, 9×10⁹.

The following examples of specific aspects for carrying out the present invention are offered for illustrative purposes only and are not intended to limit the scope of the present invention in any way.

### EXAMPLES

### Example 1: Modified ADMSCs

Native ADMSCs, characterized as CD105⁺, CD73⁺, CD90⁺, CD45⁻, CD34⁻, CD11b⁻, CD19⁻, HLA-DR⁻, CD were isolated from stromal vascular fraction of human adipose tissue. The native ADMSCs were cultured in a MSC culture medium comprising alpha-MEM (commercially available from Gibco, USA), 5% Helio Bioscience UltraGRO (commercially available from AventaCell BioMedical Corp, USA) and 2 U/ml Heparin (commercially available from MP Biomedicals, USA) and sub-cultured when the cells reached 80% confluency. Before trypsinization, MSC culture medium was removed and the cells were washed with 1×DPBS (Gibco, 14190-144). Incubated the cells with 0.25% Trypsin-EDTA (Gibco, 25200072) for 3 minutes at room temperature. Once the cells were detached, MSC culture medium was added to neutralize the trypsin. The cultured cells were transferred to a centrifuge tube and centrifuged at 330 g for 5 minutes. Resuspend the cell pellet in MSC culture medium and the cells were incubated at 37° C. in 5% CO₂ incubator.

After 12 passages, the cultured ADMSCs (hereafter modified ADMSCs) were analyzed using flow cytometry. Table 1 below shows the % of modified ADMSC that express a specific biomarker.

TABLE 1

| | | | CD45, | | | |
|---|---|---|---|---|---|---|
| | | | CD34, | | | |
| | | | CD11b, | | | |
| | | | CD19 & | | | |
| CD73 | CD90 | CD105 | HLA-DR | CD273 | CD55 | CD46 |
| 99.46% | 99.88% | 95.82% | 0.02% | 96.92% | 91.41 | 94.13% |

In addition, the modified ADMSCs retain the chondrogenic, osteogenic and adipogenic differentiation abilities.

### Example 2: Maintenance of SH-SY5Y ATXN3/Q75 Cells

The SH-SY5Y ATXN3/Q75 cells and the culture system were obtained from Dr. Guey-Jen Lee-Chen, National Normal University, Taiwan. SH-SY5Y ATXN3/Q75 cells were cultured in a medium comprising DMEM (Dulbecco's Modified Eagle's Medium)/Hams F-12 (DMEM/F12) (Corning, 16-405-CV) and supplemented with 10% FBS (Corning, 35-010-CV), 5 μg/ml blasticidin (InvivoGen, ant-bl) and 100 μg/ml hygromycin (InvivoGen, ant-hg). The SH-SY5Y ATXN3/Q75 cells were sub-cultured when the cells reached 80% confluency. Before trypsinization, the medium was removed and the cells were washed with 1×DPBS (Gibco, 14190-144) and incubated with 0.25% Trypsin-EDTA (Gibco, 25200072) for 3 minutes at room temperature. Once the cells were detached, the DMEM/F12 medium supplemented with 10% FBS, 5 μg/ml blasticidin and 100 μg/ml hygromycin was added to neutralize the trypsin. The SH-SY5Y ATXN3/Q75 cells were transferred to a centrifuge tube and centrifuged at 330 g for 5 minutes. Resuspend the cell pellet in the DMEM/F12 medium supplemented with 10% FBS, 5 μg/ml blasticidin and 100 μg/ml hygromycin and the cells were incubated at 37° C. in 5% CO₂ incubator. Within 7 days, cells will become 80% confluent and can be used for further experiments.

### Example 3: The Effect of Modified ADMSC on SCA3 Diseased Cell Model-SH-SY5Y ATXN3/Q75 Cells

Dr. GJ Lee (National Taiwan Normal University) had generated a SCA3 cell model, with the accumulation of polyQ expanded ataxin-3 to form aggregation in neuronal cells, a phenomenon commonly observed in the brains of SCA3 patients [6]. Different ratios of modified ADMSC to SH-SY5Y ATNX3/Q75 cells (0:1, 1:20, 1:15 and 1:10) were examined to determine the dose-dependent efficacy of modified ADMSC. In brief, 9×10³ modified ADMSCs, 1.2×10⁴ modified ADMSCs and 1.8×10⁴ ADMSCs were seeded into the upper compartment of a six-well transwell (Falcon, 353090) system and cultured in the MSC culture medium comprising alpha-MEM, 5% Helio Bioscience UltraGRO and 2 U/ml Heparin.

2.0×10⁵ SH-SY5Y ATXN3/Q75 cells were placed into the lower compartment of a six-well transwell system (Falcon, 353502) and culture in a medium comprising DMEM/F12, 10% FBS and 10 μM retinoic acid (Sigma, R2625). On the next day, modified ADMSC-seeded inserts were transferred to the six-well plate in the lower compartment wherein the SH-SY5Y ATXN3/Q75 cells were cultured in a medium containing DMEM/F12 and 10% FBS. SH-SY5Y ATXN3/Q75 cells were treated with 5 μg/ml doxycycline (Sigma, D9891) to induce mutant ataxin-3 expression. 5 μg/ml doxycycline and 10 μM retinoic acid were added every three days for a week.

### Example 4: The Effect of Activin a on SCA3 Diseased Cell Model (SH-SY5Y ATXN3/Q75 Cells)

The effect of different concentrations of Activin A (0.2-5 ng/ml) (Biolegened, 592002) on the SCA3 diseased cell model (SH-SY5Y ATXN3/Q75 cells) was examined. SH-SY5Y ATXN3/Q75 cells were first seeded into six-well plates cell culture dishes (Corning, 353046) at the density of 20,833 cells/cm² with DMEM/F12 medium supplemented with 10% FBS, 5 μg/ml blasticidin, 100 μg/ml hygromycin and 10 μM retinoic acid. On the next day, SH-SY5Y ATXN3/Q75 cells were treated with 5 μg/ml doxycycline and 10 μM retinoic acid and different doses (0.2, 1.0 and 5.0 ng/ml) of Activin A. 5 μg/ml doxycycline and 10 μM retinoic acid were added every three days for a week.

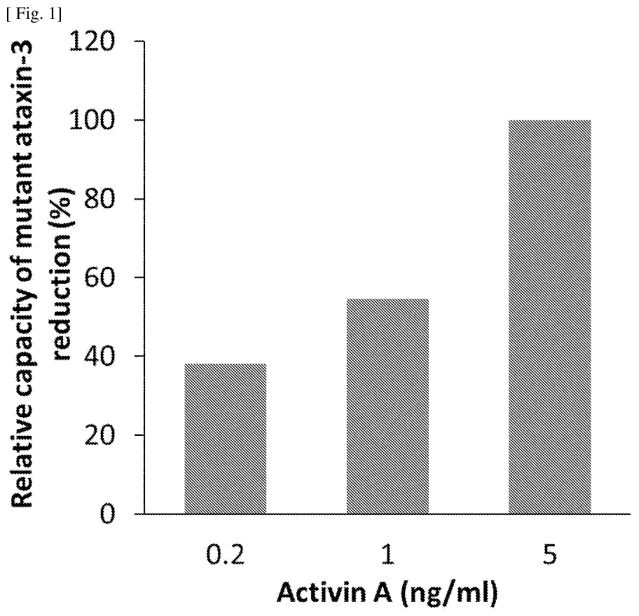

Result: Activin A reduced mutant ataxin-3 expression in SCA3 cell model. FIG. 1 shows Activin A reduces mutant ataxin-3 level in the SH-SY5Y ATXN3/Q75 cells in a dose dependent manner.

### Example 5: The Combination Effect of Modified ADMSC and Activin a on SCA3 Diseased Cell Model-SH-SY5Y ATXN3/Q75 Cells

9×10³ modified ADMSCs, 1.2×10⁴ modified ADMSCs and 1.8×10⁴ modified ADMSCs were seeded into the upper compartment of a six-well transwell system and cultured in a MSC culture medium comprising alpha-MEM, 5% Helio Bioscience UltraGRO and 2 U/ml Heparin. 2.0×10⁵ SH-SY5Y ATXN3/Q75 cells were placed into the lower compartment of a six-well transwell system and cultured in DMEM/F12 medium supplemented with 10% FBS and 10 μM retinoic acid. On the next day, modified ADMSC-seeded inserts were transferred to the six-well plate in the lower compartment wherein the SH-SY5Y ATXN3/Q75 cells were cultured in DMEM/F12 medium supplemented with 10% FBS. SH-SY5Y ATNX3/Q75 cells and modified ADMSC were treated with various concentrations of Activin A (0.2, 1.0 and 5.0 ng/ml). 5 μg/ml doxycycline and 10 μM retinoic acid were added every three days for a week.

### Pulse Shape Analysis (PulSA):

On Day 7, SH-SY5Y ATXN3/Q75 cells with different treatment conditions described above were collected and resuspended in DPBS containing 2% FBS for analysis by flow cytometry (Guava®easyCyte™). The results were analyzed by Guava software, and the level of mutant ataxin-3 was determined.

The effect of modified ADMSC and/or Activin A was assessed using the combination index (CI) method (Chou, T. C., Cancer Res, 2010.70 (2): p. 440-6).

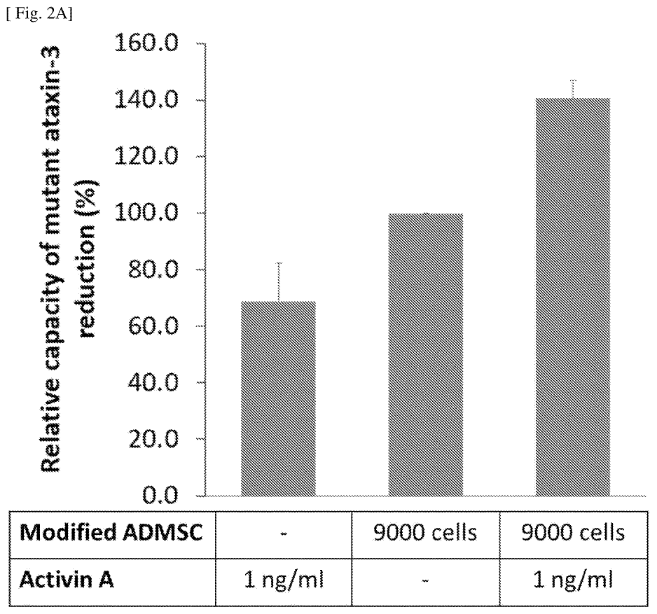

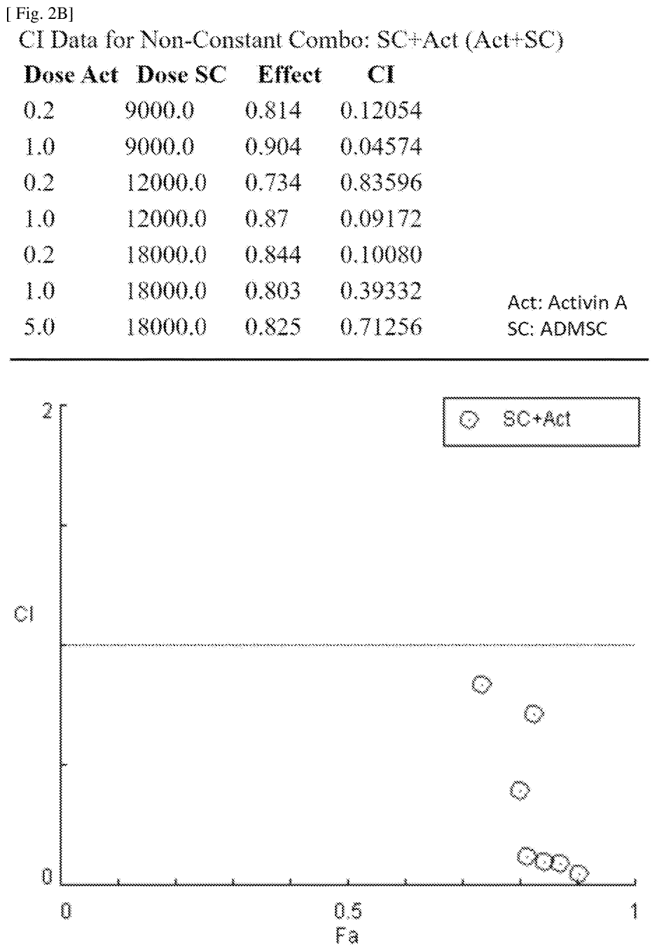

Results: FIG. 2A and FIG. 2B show the synergistic effect of the combinations of various doses of modified ADMSC and various doses of Activin A in reducing mutant ataxin-3 (The CI value is less than 1 indicates synergy).

### Example 6: Modified Adipose Tissue Derived Stem Cells (ADMSCs) that Express Activin a (Activin a Expressing ADMSC)

One day before transfection, 4.8×10⁴ modified ADMSCs were cultured in 1.5 ml of MSC culture medium comprising alpha-MEM, 5% Helio Bioscience UltraGRO and 2 U/ml Heparin per well so the modified ADMSCs reached 50-60% confluent at the time of transfection. Diluted plasmid DNA, Activin A expression plasmid (Sino Biological, HG10429-UT) or control plasmid (Sino Biological, CV011), and Lipofectamine stem (Thermo Fisher Scientific, STEM00001) in Opti-MEM I Medium (Thermo Fisher Scientific, 11058021) were prepared separately in difference eppendorf and incubated for 5 minutes at room temperature. After incubation, diluted plasmid DNA was combined with diluted Lipofectamine stem, mixed gently and incubated for 10 minutes at room temperature to allow the plasmid DNA-Lipofectamine stem complexes to form. 150 μl of plasmid DNA-Lipofectamine stem complexes was added to each well containing modified ADMSCs and MSC culture medium and the mixture was mixed gently by rocking the plate back and forth. Incubate the cells at 37° C. in a CO₂ incubator for 24 hours. Then Activin A expressing ADMSCs were replenished with MSC culture medium. Activin A expressing ADMSCs were cultured in standard conditions (5% CO₂; 37° C.) for 24 hr. The conditioned medium samples were collected, centrifuged to remove cell debris, then frozen in aliquots at −20° C.

The effect of conditioned medium in SCA3 diseased cell model-SH-SY5Y ATXN3/Q75 cells:

SH-SY5Y ATXN3/Q75 cells were treated with different doses of conditioned medium, secreted from Activin A expressing ADMSC described above and analyzed for mutant ataxin-3 expression level.

First, SH-SY5Y ATXN3/Q75 cells were seeded into six-well plates cell culture dishes at the density of 20,833 cells/cm² with DMEM/F12 medium supplemented with 10% FBS, 5 μg/ml blasticidin, 100 μg/ml hygromycin and 10 μM retinoic acid. On the next day, SH-SY5Y ATXN3/Q75 cells were treated with 5 μg/ml doxycycline and 10 μM retinoic acid and different doses (3%, 10%, 30%, 50% and 70%) of conditioned medium. 5 μg/ml doxycycline and 10 μM retinoic acid were added every three days for a week. On Day 7, SH-SY5Y ATXN3/Q75 cells with different treatment conditions were collected and analysis by flow cytometry.

Results: Overexpression of Activin A in modified ADMSCs enhanced their ability in reducing mutant ataxin-3 expression in a SCA3 cell model.

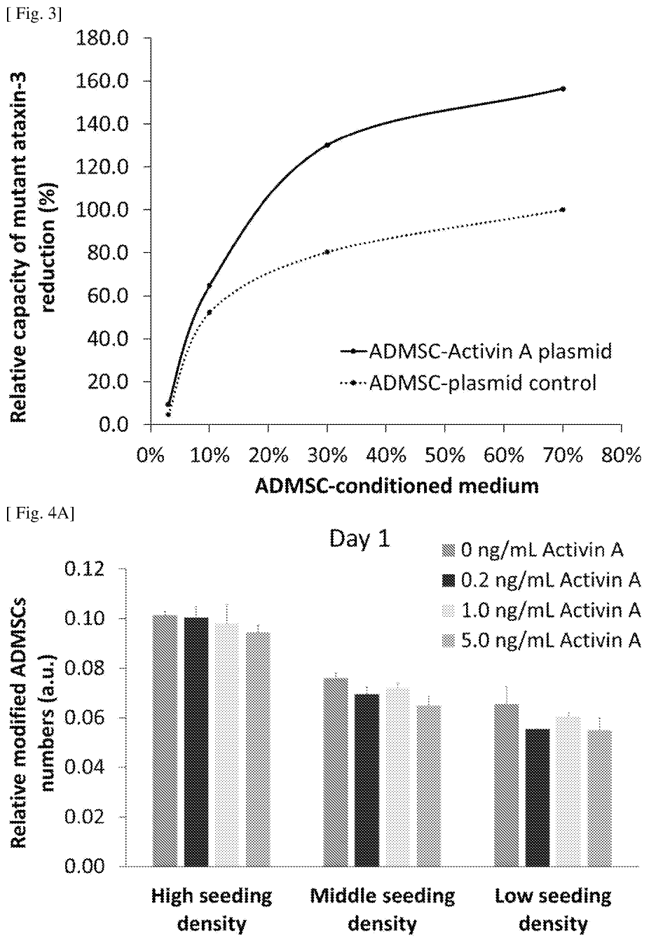

SH-SY5Y ATXN3/Q75 cells were treated with different volume fraction of Activin A expressing ADMSC conditioned medium. FIG. 3 shows that conditioned medium from Activin A expressing ADMSCs reduces mutant ataxin-3 expression in a dose dependent manner, compared to that of plasmid control group.

### Example 7: The Effect of Activin a on the Modified Adipose Tissue Derived Stem Cells (ADMSCs)

To effect of Activin A on the proliferation of modified ADMSCs was assessed.

### Method:

The proliferation of ADMSC was tested by using CCK-8 (DOJINDO, Japan). Cells were seeded into the wells of a 96-well microtiter plate and incubated at 37° C. for 16~18 hours, replacing fresh culture medium containing Activin A at different concentrations to culture at 37° C. for 3 days. CCK-8 solution (10 μL per well) was added, followed by an additional incubation for 3 hours. The absorbance was measured at 450 nm. High seeding density: 4, 762 cells/cm² (equivalent to the co-culture ratio of modified ADMSCs and SH-SY5Y ATNX3/Q75=1:10 in the present application); Middle seeding density: 3, 175 cells/cm² (equivalent to the co-culture ratio of modified ADMSCs and SH-SY5Y ATNX3/Q75=1:15); Low seeding density: 2, 381 cells/cm² (equivalent to the co-culture ratio of modified ADMSCs and SH-SY5=1:20).

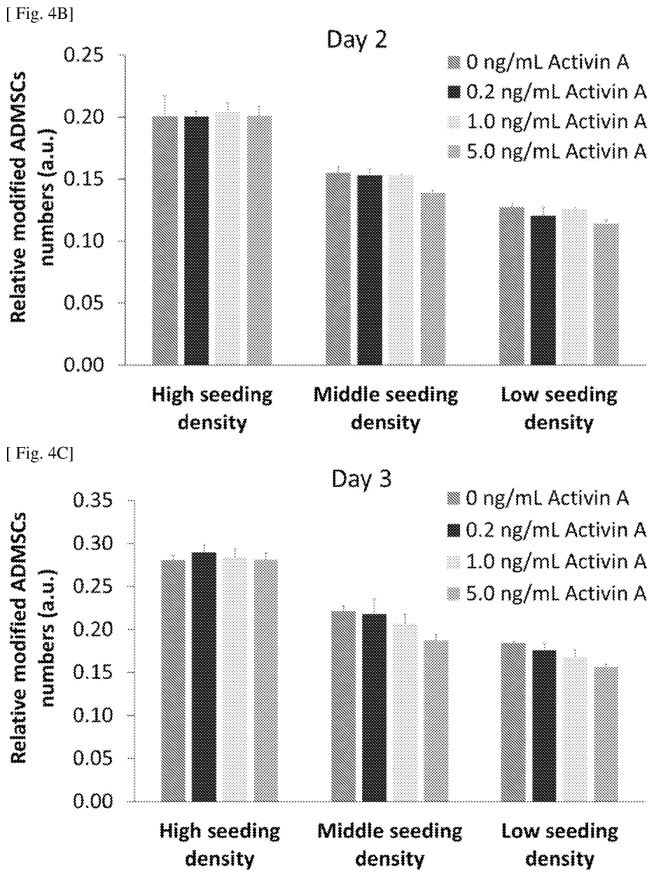

Results: FIG. 4A to 4C show different doses of Activin A do not affect the proliferation of modified ADMSCs at different densities. These data confirm that the synergistic efficacy of combined modified ADMSCs and activin A to reduce mutant ataxin-3 levels is not due to an increased number of modified ADMSCs.

### Example 8: Characterization of Native ADMSCs

A population (1.00×10⁸) of stromal vascular fraction cells were isolated from adipose tissue of a healthy adult donor and processed as follows:

Harvested stromal vascular fraction was first centrifuged for 3-10 minutes at 100 RCF to remove the unwanted watery blood, and digested with collagenase for 0.5-6 hours at 37° C. The length of the aforementioned incubation time and appended shaking depend on the tissue condition and size. The isolated native ADMSCs were then washed with PBS and centrifuged for 3-10 minutes at 400 RCF twice. After dispersion of the pellet, isolated native ADMSCs were counted and plated on cell culture flasks at a density of lower than 5×10⁶ cells/cm². The surface markers of the native ADMSCs in the stromal vascular fraction, without any further culture, were analyzed using flow cytometry analysis.

Region of positive/specific staining was set up using isotype control. Briefly, non-relevant immunoglobulin labeled with similar fluorochrome as the staining antibody identifies nonspecific staining to set up a region of positive/specific staining. In the present study, 102 is set to be the positive threshold set up by isotype control.

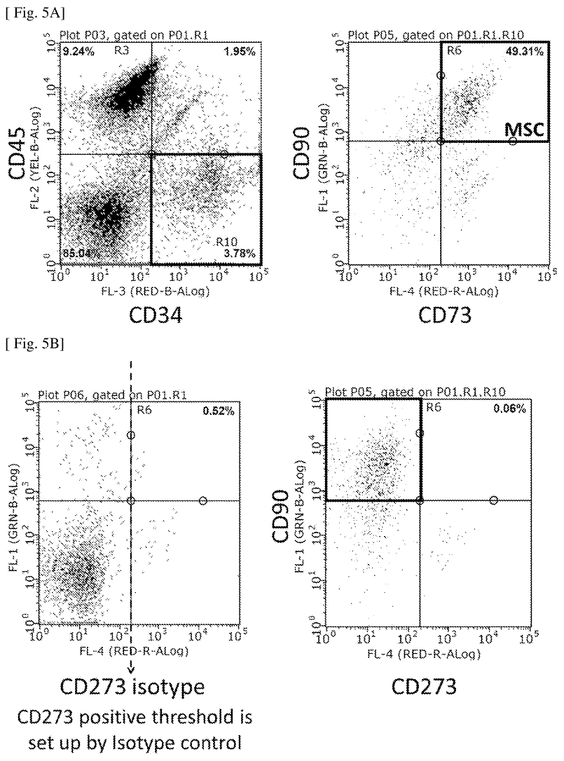

Based on the flow cytometry images, native ADMSCs express CD45⁻CD34⁺ CD90⁺ CD73⁺ expression, which is the characteristic of the native ADMSCs, see FIG. 5A.

Results: The expression of CD273 of the native ADMSCs was further examined using flow cytometry. FIG. 5B illustrates the native ADMSCs do not express CD273 (i.e., CD273).

### REFERENCES

1. McLoughlin, H. S., L. R. Moore, and H. L. Paulson, Pathogenesis of SCA3 and implications for other polyglutamine diseases. Neurobiol Dis, 2020. 134: p. 104635.
2. Rüb, U., et al., Clinical features, neurogenetics and neuropathology of the polyglutamine spinocerebellar ataxias type 1, 2, 3, 6 and 7. Prog Neurobiol, 2013. 104: p. 38-66.
3. Da Silva, J. D., A. Teixeira-Castro, and P. Maciel, From Pathogenesis to Novel Therapeutics for Spinocerebellar Ataxia Type 3: Evading Potholes on the Way to Translation. Neurotherapeutics, 2019. 16 (4): p. 1009-1031.
4. Morianos, I., et al., Activin-A in the regulation of immunity in health and disease. Journal of Autoimmunity, 2019. 104: p. 102314.
5. Lin, C. H., et al., Novel Lactulose and Melibiose Targeting Autophagy to Reduce PolyQ Aggregation in Cell Models of Spinocerebellar Ataxia 3. CNS Neurol Disord Drug Targets, 2016. 15 (3): p. 351-9.

1-12. (canceled)

13. A pharmaceutical composition, comprising:
(a) Activin A; and
(i) Modified adipose tissue derived stem cells (modified ADMSCs) that express CD273, CD46, CD55, CXCR4, CD105, CD73, and CD90 but not CD45, CD34, CD11b, CD19 and HLA-DR, or
(ii) Activin A expressing ADMSCs that express CD273, CD46, CD55, CXCR4, CD105, CD73, CD90 and Activin A but not CD45, CD34, CD11b, CD19 and HLA-DR.

14. A method for treating polyQ disease, comprising the step of administering a therapeutic effective amount of the pharmaceutical composition of claim 13.

15. The method of claim 14, wherein said polyQ disease is spinocerebellar ataxias (SCA), Machado-Joseph disease (MJD/SCA3), Huntington's disease (HD), dentatorubral pallidoluysian atrophy (DRPLA) or spinal and bulbar muscular atrophy, X-linked 1 (SMAX1/SBMA).

16. The method of claim 15, wherein said SCA is SCA3.

17. The method of claim 15, wherein said SCA is SCA6.

18. A method for reducing mutant polyQ protein in a subject, comprising the step of administering a therapeutic effective amount of the pharmaceutical composition of claim 13.

19. The method of claim 18, wherein the polyQ protein is ATXN-3.
